(19) United States
(12) Patent Application Publication
(10) Pub. No.: US 2021/0401319 A1
(43) Pub. Date: Dec. 30, 2021

(54) SCREENING APPARATUS USING MULTI-CHANNEL ARRAY ELECTRODE PROBE AND METHOD OF OPERATING SCREENING APPARATUS

(71) Applicants: UNIVERSITY-INDUSTRY COOPERATION GROUP OF KYUNG HEE UNIVERSITY, Yongin-si, Gyeonggi-do (KR); THE CATHOLIC UNIVERSITY OF KOREA INDUSTRY-ACADEMIC COOPERATION FOUNDATION, Seoul (KR)

(72) Inventors: Dong In OH, Seoul (KR); Eung Je WOO, Seongnam-shi (KR); Dong Choon PARK, Suwon-si (KR); Seung Geun YEO, Seoul (KR)

(73) Assignees: UNIVERSITY-INDUSTRY COOPERATION GROUP OF KYUNG HEE UNIVERSITY, Yongin-si, Gyeonggi-do (KR); THE CATHOLIC UNIVERSITY OF KOREA INDUSTRY-ACADEMIC COOPERATION FOUNDATION, Seoul (KR)

(21) Appl. No.: 17/294,545

(22) PCT Filed: Nov. 13, 2019
(86) PCT No.: PCT/KR2019/015390

§ 371 Date: May 17, 2021

(30) Foreign Application Priority Data
Nov. 16, 2018 (KR) 10-2018-0141644

(51) Int. Cl. A61B 5/0536; A61B 1/04; A61B 5/0538; A61B 5/00; A61B 1/00

(52) U.S. Cl. CPC A61B 5/0536; A61B 1/04; A61B 5/4331; A61B 5/7425; A61B 1/0005; A61B 5/0538

(57) ABSTRACT

Disclosed are a screening apparatus using a multi-channel array electrode probe and a method of operating the screening apparatus, wherein the screening apparatus includes a probe inserted into the inside of the human body and including a plurality of electrodes in contact with any one portion of the inside and an endoscope camera for photographing the portion; a location identifier for identifying a location in contact with the electrodes in an image photographed by the endoscope camera; an electrical property measuring device for measuring the electrical properties of the identified location; a mapping processor for mapping the measured electrical properties to an electrical model for the internal tissue; and a visualization information generator for, based on the mapping results, generating visualization information for visualization of the degree of deformation of the tissue with respect to the measured electrical properties.

### TECHNICAL FIELD

The present invention relates to a technical idea of screening the inside of the human body using a multi-channel array electrode probe. More particularly, the present invention relates to a screening apparatus using a multi-channel array electrode probe and a method of operating the screening apparatus. According to the present invention, screening of the entire cervix may be performed using electrodes and an endoscope camera included in a probe capable of being inserted into the human body.

### BACKGROUND ART

Conventionally, when examining the entire cervix or diagnosing a cervix-related disease, cytology (e.g., Pap testing) or partial biopsy has been performed.

In general, cytology is inexpensive. However, since cytology is based on a smear test performed on exfoliated cells, cytology has a high false negative rate. In addition, it is impossible to predict the location or depth of a lesion through cytology.

In addition, partial biopsy is more accurate than cytology. However, since an examiner performs a medical examination only on tissues with suspicious medical findings, an experienced examiner with long clinical experience is required to lower the examiner's bias.

Accordingly, since cervical dysplasia occurs locally, despite the presence of the disease, the disease may not be found upon examination, or diagnosis accuracy may vary depending on examiners.

Since diagnosis at the time of colposcopy depends on the subjective judgment of an examiner, a bias may exist in the test results. In the case of conization, group examination is impossible. Accordingly, these tests may affect procedure cost, pregnancy, and childbirth.

In addition, in the case of early stage cervical dysplasia, a lesion may be manipulated using high frequency or laser. However, since tissue examination is impossible, it is difficult to accurately diagnose and confirm the existence of residual diseases.

The size of the global cervical cancer diagnosis market in 2015 was 7.4 trillion won, showing a growth rate of 5.9%.

Cervical cancer is the second most common female cancer in the world, and is one of the most well-understood and preventable cancers among major human malignancies.

According to the American Cancer Society, 12,820 cases of cervical cancer were diagnosed in 2017, and 4,210 people were predicted to die due to cervical cancer.

In high-income countries, diagnosis of cervical cancer is performed on the basis of cervical cytology screening. Accordingly, in these countries, cervical cancer is diagnosed and prevented more effectively using expensive methods.

However, nearly 90% of deaths due to cervical cancer occur in low-income, developing countries.

Accordingly, there is a need to develop a diagnostic tool that is inexpensive and capable of simple examination.

### DISCLOSURE

### Technical Problem

Therefore, the present invention has been made in view of the above problems, and it is one object of the present invention to screen the entire area of the cervix to examine normal tissues, cancer tissues, or atypical tissues at a portion accessible through an orifice of the human body.

It is another object of the present invention to reduce the cost of screening for cervical dysplasia and cervical cancer and increase measurement accuracy by acquiring integrated data using a multi-channel array electrode and an endoscope camera.

It is still another object of the present invention to quantify the degree of penetration by region and depth by using a multi-channel array electrode and an endoscope camera.

It is still another object of the present invention to increase, by adjusting the distance between a selected electrode pair, measurement sensitivity for a site in a tissue at which dysplasia is observed.

It is still another object of the present invention to support real-time diagnosis and patient-specific treatment by imaging the electrical properties of tissues at a portion accessible through an orifice of the human body.

It is still another object of the present invention to reduce the mental and physical burden of a patient by simplifying and expediting a process from examination to diagnosis.

It is still another object of the present invention to accurately inspect a site where dysplasia occurs based on image data acquired by an endoscope camera and measurement results for electrical properties.

It is still another object of the present invention to form a greater number of impedance spectrum measurement points than installed electrodes based on the geometric position of an injection current channel and a voltage measurement channel, and to obtain a map with improved resolution by generating interpolated data based on sensitivity measurement analysis between the measurement points by using the similarity between the positions of impedance spectra measured at each measurement point.

It is yet another object of the present invention to increase the probability of detecting cervical dysplasia and cervical cancer by improving the efficiency of designating a biopsy area by providing all electrical property values and a map of electrical property change by area.

### Technical Solution

In accordance with one aspect of the present invention, provided is a screening apparatus using a multi-channel array electrode probe, including a probe inserted inside of a human body and including a plurality of electrodes in contact with any one portion of the inside and an endoscope camera for photographing the portion; a location identifier for identifying a location in contact with the electrodes in an image photographed by the endoscope camera; an electrical property measuring device for measuring electrical properties of the identified location; a mapping processor for mapping the measured electrical properties to an electrical model for the internal tissue; and a visualization information generator for, based on the mapping results, generating visualization information for visualization of a degree of deformation of the tissue with respect to the measured electrical properties.

According to one embodiment of the present invention, based on similarity between an impedance spectrum of a first measurement point and an impedance spectrum of a second measurement point among a plurality of measurement points located under the probe, the electrical property measuring device may generate data about an impedance spectrum between the first measurement point and the second measurement point.

According to one embodiment of the present invention, the mapping processor may generate impedance spectrum distribution data based on a plurality of impedance spectra related to the measured electrical properties, and may map a probability distribution between normal tissue and atypical tissue to an electrical model for the internal tissue using frequency difference in the impedance spectrum distribution data.

According to one embodiment of the present invention, the electrical property measuring device may determine a measurement sensitivity distribution for the portion according to a location of an electrode pair selected from the electrodes.

According to one embodiment of the present invention, the screening apparatus may further include a probe controller for determining an electrode pair for collecting any one of current and voltage from the electrodes based on the determined measurement sensitivity distribution.

According to one embodiment of the present invention, to adjust a distance between the electrodes, the probe controller may change a combination of electrodes constituting the electrode pair, and may integrate and analyze a plurality of measurement data to increase measurement sensitivity.

According to one embodiment of the present invention, the probe controller may control the probe to measure an electrical impedance spectrum by selecting any one electrode from the electrode pair and supplying any one of current and voltage to the electrode, and by collecting any one of current and voltage through the remaining electrode of the electrode pair.

According to one embodiment of the present invention, the electrical property measuring device may non-invasively measure electrical properties of the internal tissue by measuring an electrical impedance spectrum of the internal tissue through the electrodes.

According to one embodiment of the present invention, the portion may include a portion accessible through an orifice of the human body, and the accessible portion may include at least one of a vagina, a cervix, a rectum, an esophagus, and a stomach.

According to one embodiment of the present invention, the screening apparatus may further include a display controller for controlling a display to output the generated visualization information, wherein the generated visualization information represents a degree of deformation of the tissue through difference in color, shade, shape, range, or distribution.

In accordance with another aspect of the present invention, provided is a method of operating a screening apparatus using a multi-channel array electrode probe, wherein the screening apparatus includes a probe inserted inside of a human body and including a plurality of electrodes in contact with any one portion of the inside and an endoscope camera for photographing the portion, and the method includes a step of identifying, in a location identifier, a location in contact with the electrodes in an image photographed by the endoscope camera; a step of measuring, in an electrical property measuring device, electrical properties of the identified location; a step of mapping, in a mapping processor, the measured electrical properties to an electrical model for the internal tissue; and a step of generating, in a visualization information generator, visualization information for visualization of a degree of deformation of the tissue with respect to the measured electrical properties based on the mapping results.

According to one embodiment of the present invention, the step of measuring electrical properties may include a step of determining a measurement sensitivity distribution for the portion according to a location of an electrode pair selected from the electrodes; a step of determining, in a probe controller, an electrode pair for collecting any one of current and voltage from the electrodes based on the determined measurement sensitivity distribution; and a step of changing, in the probe controller, a combination of electrodes constituting the electrode pair to adjust a distance between the electrodes, and integrating and analyzing a plurality of measurement data to increase measurement sensitivity.

According to one embodiment of the present invention, the step of determining an electrode pair for collecting any one of current and voltage may include a step of selecting any one electrode from the electrode pair and supplying any one of current and voltage to the electrode; and a step of controlling the probe to measure an electrical impedance spectrum by collecting any one of current and voltage through the remaining electrode of the electrode pair.

According to one embodiment of the present invention, the method may further include a step of controlling, in a display controller, a display to output the generated visualization information, wherein the generated visualization information represents a degree of deformation of the tissue through difference in color, shade, shape, range, or distribution.

According to one embodiment of the present invention, the step of controlling a display to output the generated visualization information may include a step of controlling the display to output any one of a location, a depth and a degree of progression of atypical tissue (dysplasia) through the difference in color, shade, shape, range, or distribution.

### Advantageous Effects

The present invention can screen the entire area of the cervix to examine normal tissues, cancer tissues, or atypical tissues at a portion accessible through an orifice of the human body.

The present invention can reduce the cost of screening for cervical dysplasia and cervical cancer and increase measurement accuracy by acquiring integrated data using a multi-channel array electrode and an endoscope camera.

The present invention can quantify the degree of penetration by region and depth by using a multi-channel array electrode and an endoscope camera.

The present invention can increase, by adjusting the distance between a selected electrode pair, measurement sensitivity for a site in a tissue at which dysplasia is observed.

The present invention can support real-time diagnosis and patient-specific treatment by imaging the electrical properties of tissues at a portion accessible through an orifice of the human body.

The present invention can reduce the mental and physical burden of a patient by simplifying and expediting a process from examination to diagnosis.

The present invention can accurately inspect a site where dysplasia occurs based on image data acquired by an endoscope camera and measurement results for electrical properties.

The present invention can form a greater number of impedance spectrum measurement points than installed electrodes based on the geometric position of an injection current channel and a voltage measurement channel, and can obtain a map with improved resolution by generating interpolated data based on sensitivity measurement analysis between the measurement points by using the similarity between the positions of impedance spectra measured at each measurement point.

The present invention can increase the probability of detecting cervical dysplasia and cervical cancer by improving the efficiency of designating a biopsy area by providing all electrical property values and a map of electrical property change by area.

### DESCRIPTION OF DRAWINGS

FIG. 1 is a block diagram showing the components of a screening apparatus according to one embodiment of the present invention.

FIG. 2 illustrates a probe according to one embodiment of the present invention.

FIG. 3 is a drawing for explaining an electrical property image according to one embodiment of the present invention.

FIGS. 4A to 4C are drawings for explaining identification of the depth and location of atypical tissue based on electrical property images according to one embodiment of the present invention.

FIGS. 5A and 5B are drawings for explaining identification of the location of atypical tissue based on electrical property images according to one embodiment of the present invention.

FIG. 6 is a graph for explaining measurement sensitivity versus specificity according to one embodiment of the present invention.

FIGS. 7 and 8 are flowcharts for explaining methods of operating a screening apparatus according to one embodiment of the present invention.

### BEST MODE

Hereinafter, various embodiments of the present invention will be described in detail with reference to the accompanying drawings.

However, it should be understood that the present invention is not limited to the embodiments according to the concept of the present invention, but includes changes, equivalents, or alternatives falling within the spirit and scope of the present invention.

In the following description of the present invention, detailed description of known functions and configurations incorporated herein will be omitted when it may make the subject matter of the present invention unclear.

In addition, the terms used in the specification are defined in consideration of functions used in the present invention, and can be changed according to the intent or conventionally used methods of clients, operators, and users. Accordingly, definitions of the terms should be understood on the basis of the entire description of the present specification.

In description of the drawings, like reference numerals may be used for similar elements. The singular expressions in the present specification may encompass plural expressions unless clearly specified otherwise in context.

In this specification, expressions such as “A or B” and “at least one of A and/or B” may include all possible combinations of the items listed together.

Expressions such as “first” and “second” may be used to qualify the elements irrespective of order or importance, and are used to distinguish one element from another and do not limit the elements.

It will be understood that when an element (e.g., first) is referred to as being “connected to” or “coupled to” another element (e.g., second), it may be directly connected or coupled to the other element or an intervening element (e.g., third) may be present.

As used herein, “configured to” may be used interchangeably with, for example, “suitable for”, “ability to”, “changed to”, “made to”, “capable of”, or “designed to” in terms of hardware or software.

In some situations, the expression “device configured to” may mean that the device “may do ˜” with other devices or components.

For example, in the sentence “processor configured to perform A, B, and C”, the processor may refer to a general purpose processor (e.g., CPU or application processor) capable of performing corresponding operation by running a dedicated processor (e.g., embedded processor) for performing the corresponding operation, or one or more software programs stored in a memory device.

In addition, the expression “or” means “inclusive or” rather than “exclusive or”. That is, unless mentioned otherwise or clearly inferred from context, the expression “x uses a or b” means any one of natural inclusive permutations.

Terms, such as “unit” or “module”, etc., should be understood as a unit that processes at least one function or operation and that may be embodied in a hardware manner, a software manner, or a combination of the hardware manner and the software manner.

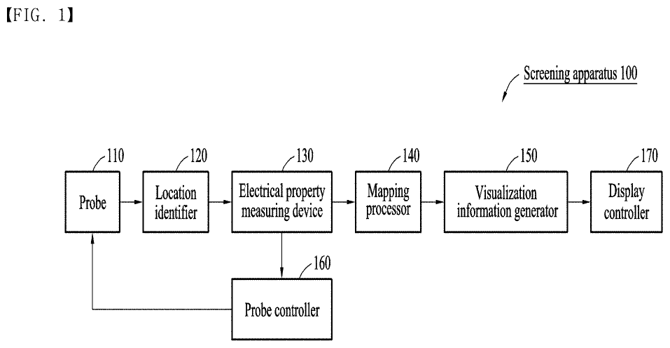

FIG. 1 is a block diagram showing the components of a screening apparatus according to one embodiment of the present invention.

Referring to FIG. 1, a screening apparatus 100 may include a probe 110, a location identifier 120, an electrical property measuring device 130, a mapping processor 140, and a visualization information generator 150.

According to one embodiment of the present invention, the probe 110 may be inserted into the human body through an orifice of the human body.

For example, the probe 110 may include a plurality of electrodes in contact with a specific internal portion of the human body and an endoscope camera for photographing the portion in contact with the electrodes. For example, the electrodes may include a multi-channel array electrode.

According to one embodiment of the present invention, the probe 110 may collect data for predicting the location and the degree of progression of cervical dysplasia and early cervical cancer.

For example, the specific internal portion may include a portion accessible through an orifice of the human body. In this case, the accessible portion may include at least one of the vagina, the cervix, the rectum, the esophagus, and the stomach.

Accordingly, the present invention may screen the entire area of the cervix to examine normal tissues, cancer tissues, or atypical tissues at a portion accessible through an orifice of the human body.

That is, according to the present invention, the entire area of the cervix may be screened by rotating a portion of an array electrode included in the probe 110. In this case, the array electrode may include a plurality of electrodes.

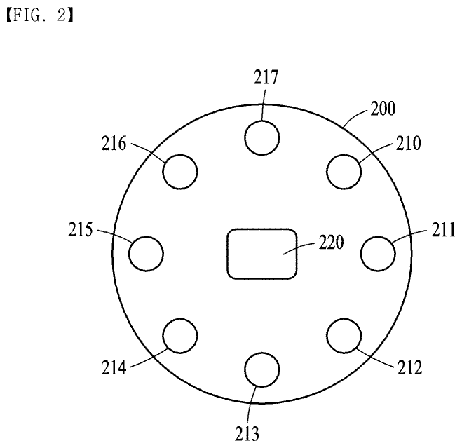

The structure of the probe 110 according to one embodiment of the present invention will be described in more detail with reference to FIG. 2.

According to one embodiment of the present invention, the location identifier 120 may identify a location in contact with a plurality of electrodes in an image photographed by the endoscope camera.

That is, the location identifier 120 may collect image data about a location probed through a plurality of electrodes, and may identify a location in contact with the electrodes by comparing the collected image data with pre-stored image data.

Accordingly, the present invention may reduce the cost of screening for cervical dysplasia and cervical cancer and increase measurement accuracy by acquiring integrated data using a multi-channel array electrode and an endoscope camera.

In addition, the present invention may quantify the degree of penetration by region and depth by using a multi-channel array electrode and an endoscope camera.

In addition, the location identifier 120 may identify a location in contact with a plurality of electrodes by comparing electrical properties measured through the electrodes with an image photographed by the endoscope camera.

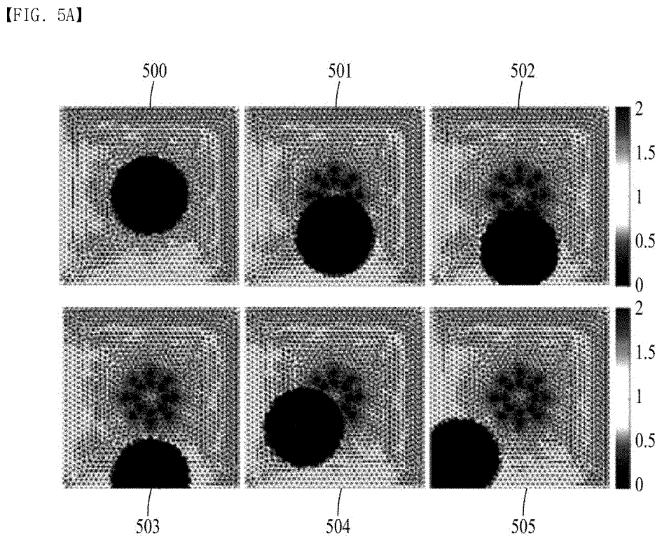

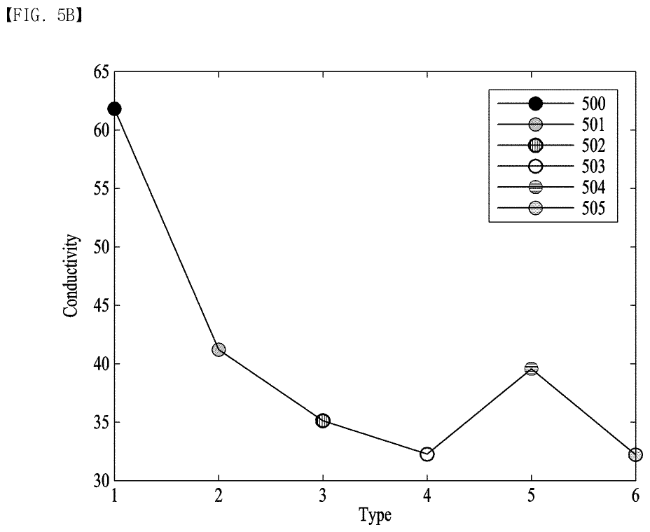

According to one embodiment of the present invention, the location identifier 120 may identify a location based on the visualization information of the measured electrical properties. A configuration for identifying a location in contact with a plurality of electrodes based on the measured electrical properties will be further described with reference to FIGS. 5A and 5B.

Accordingly, the present invention may accurately inspect a site where dysplasia occurs based on image data acquired by an endoscope camera and measurement results for electrical properties.

In addition, the present invention may reduce the cost of screening for cervical dysplasia and cervical cancer and increase measurement accuracy by acquiring integrated data using a multi-channel array electrode and an endoscope camera.

According to one embodiment of the present invention, the electrical property measuring device 130 may measure the electrical properties of a location in contact with a plurality of electrodes.

That is, the electrical property measuring device 130 may identify a location based on image data acquired by the endoscope camera, and may measure the electrical properties of a location in contact with a plurality of electrodes.

For example, the electrical property measuring device 130 may measure the electrical properties of an internal tissue non-invasively by measuring the electrical impedance spectrum of a tissue inside of the human body through a plurality of electrodes.

According to one embodiment of the present invention, the electrical property measuring device 130 may determine a measurement sensitivity distribution for the portion according to the location of an electrode pair selected from the electrodes.

For example, the electrical property measuring device 130 may measure electrical properties by measuring an electrical impedance spectrum for any one of current and voltage that are supplied through any one electrode of an electrode pair and collected through the remaining electrode.

According to one embodiment of the present invention, the electrical property measuring device 130 may interpolate the impedance spectrum between measurement points based on the similarity of impedance spectra.

That is, based on the similarity between the impedance spectrum of a first measurement point and the impedance spectrum of a second measurement point among a plurality of measurement points located under a probe, the electrical property measuring device 130 may generate data about the impedance spectrum between the first measurement point and the second measurement point.

That is, the electrical property measuring device 130 may also obtain data about the impedance spectrum of a portion positioned under the probe or a portion where the electrode is not positioned.

For example, the electrical property measuring device 130 may derive a plurality of measurement combinations while changing the measurement interval between a plurality of electrodes, and may change a measurement combination according to the derived measurement combinations.

For example, when voltage or current is supplied through a first electrode and voltage or current is measured through a third electrode, a second electrode located between the first electrode and the third electrode may be skipped.

In addition, when the second electrode is skipped, since current or voltage is spatially spread more widely inside a measurement target, the degree to which the voltage decreases is relatively small.

Accordingly, when the difference between the maximum voltage and the minimum voltage increases relatively and a voltage range increases, since the number of voltages capable of being distinguished for noise increases, image quality may be improved.

However, as the number of skipped electrodes decreases, sensitivity for change in an impedance spectrum increases. Accordingly, when an image is restored, an integrated high-resolution image may be restored in consideration of a sensitivity distribution according to the locations of several electrode pairs.

According to one embodiment of the present invention, the mapping processor 140 may map a measured impedance spectrum to the electrical model of an internal tissue.

For example, the electrical model of an internal tissue may be obtained using pre-stored data about the electrical properties of normal tissue.

For example, the mapping processor 140 may visualize electrical properties analyzed in an electrical model, thereby increasing discrimination power for tissue differences.

According to one embodiment of the present invention, the mapping processor 140 may generate impedance spectrum distribution data based on a plurality of impedance spectra related to electrical properties, and may map a probability distribution between normal tissue and atypical tissue to the electrical model of an internal tissue using frequency difference in the impedance spectrum distribution data.

That is, the mapping processor 140 may probabilistically map the distribution of normal tissue and atypical tissue using the frequency difference between impedance spectra in impedance spectrum distribution data.

According to one embodiment of the present invention, the mapping processor 140 may check the characteristics of an internal tissue by unwrapping the layer of a measurement target from data measured according to an interval in electrode combination.

For example, the mapping processor 140 may measure an impedance spectrum between a first electrode and a second electrode, may measure an impedance spectrum between the first electrode and a third electrode when the distance between the third electrode and the first electrode is longer than the distance between the second electrode and the first electrode, and may obtain internal depth information for a region located between the first electrode and the third electrode from the difference between the measured impedance spectra.

The present invention may form a greater number of conductivity measurement points than installed electrodes based on the geometric position of an injection current channel and a voltage measurement channel, and may obtain a map with improved resolution by generating interpolated data based on sensitivity measurement analysis between the measurement points by using the similarity between the positions of impedance spectra measured at each measurement point.

According to one embodiment of the present invention, the visualization information generator 150 may generate visualization information for visualizing the degree of deformation of a tissue with respect to measured electrical properties as a result of mapping.

For example, the visualization information generator 150 may generate visualization information indicating change in electrical properties based on mapping results.

For example, visualization information may include an electrical property measurement image and the quantified probability data of cervical dysplasia and early cervical cancer.

According to another embodiment of the present invention, the screening apparatus 100 may further include a probe controller 160 and a display controller 170.

For example, the probe controller 160 may select an electrode pair for collecting any one of current and voltage from a plurality of electrodes based on measurement sensitivity.

In addition, to adjust the distance between electrodes, the probe controller 160 may change a combination of electrodes constituting an electrode pair, and may integrate and analyze a plurality of measurement data to increase measurement sensitivity.

Accordingly, according to the present invention, by adjusting the distance between an electrode pair, measurement sensitivity for a point where dysplasia occurs in a tissue may be increased.

For example, the probe controller 160 may change the distance between electrodes by changing any one of electrodes selected as an electrode pair.

For example, the probe controller 160 may control the probe 110 to select any one electrode from an electrode pair and supply any one of current and voltage.

In addition, the probe controller 160 may control the probe 110 to collect any one of current and voltage through the remaining electrode of the electrode pair and measure an electrical impedance spectrum.

According to one embodiment of the present invention, the probe controller 160 may control the coefficients of voltage components for a plurality of electrodes.

For example, the probe controller 160 may determine measurement sensitivity based on a measured impedance spectrum, and may control the coefficients of voltage components for electrodes.

For example, the probe controller 160 may reduce the coefficients of voltage components for electrodes corresponding to the peripheral region of a measurement point among a plurality of electrodes and having relatively low measurement sensitivity, and may increase the coefficients of voltage components for electrodes corresponding to the central region of a measurement point among a plurality of electrodes and having relatively high measurement sensitivity.

For example, the display controller 170 may control a display to output visualization information.

In addition, the display controller 170 may control the display to output any one of the location, the depth, and the degree of progression of atypical tissue (dysplasia) through difference in color, shade, shape, range, or distribution.

Accordingly, the present invention may reduce the mental and physical burden of a patient by simplifying a process from examination to diagnosis.

In addition, the present invention may accurately inspect a site where dysplasia occurs based on image data acquired by an endoscope camera and measurement results for electrical properties.

FIG. 2 illustrates a probe according to one embodiment of the present invention.

Referring to FIG. 2, a probe 200 according to one embodiment of the present invention may include a plurality of electrodes 210, 211, 212, 213, 214, 215, 216, and 217 having an array structure and an endoscope camera 220.

According to one embodiment of the present invention, the electrodes 210, 211, 212, 213, 214, 215, 216, and 217 having an array structure may form electrode pairs according to combination.

For example, the electrodes may include a first electrode 210, a second electrode 211, a third electrode 212, a fourth electrode 213, a fifth electrode 214, a sixth electrode 215, a seventh electrode 216, and an eighth electrode 217.

For example, the first electrode 210 and the second electrode 211 may be selected as an electrode pair for collecting any one of current and voltage.

For example, any one of current and voltage may be applied to the first electrode 210, and any one of current and voltage may be collected through the second electrode 211.

In the above description, although an embodiment in which the electrodes 210, 211, 212, 213, 214, 215, 216, and 217 are configured as a single ring or layer has been described, but the electrodes 210, 211, 212, 213, 214, 215, 216, and 217 may be configured as a plurality of rings or layers including a plurality of electrodes.

Accordingly, the number of the electrodes may be increased according to the number of rings or layers.

According to one embodiment of the present invention, the probe may be in contact with any one portion inside the human body, and the probe controller may select the first electrode 210 and the seventh electrode 216 as an electrode pair.

In addition, the electrical property measuring device may measure an electrical impedance spectrum based on impedance data collected using the first electrode 210 and the seventh electrode 216, and may measure the electrical properties of any one portion based on the measured electrical impedance spectrum.

For example, when the first electrode 210 and the seventh electrode 216 constitute an electrode pair, to adjust the distance between electrodes, the probe controller may reduce the distance by changing an electrode corresponding to the first electrode 210 from the seventh electrode 216 to the eighth electrode 217.

In addition, when the first electrode 210 and the seventh electrode 216 constitute an electrode pair, to adjust the distance between electrodes, the probe controller may increase the distance by changing an electrode corresponding to the first electrode 210 from the seventh electrode 216 to the sixth electrode 215.

According to one embodiment of the present invention, the endoscope camera 220 may collect image data by photographing a portion in contact with the first electrode 210 and the seventh electrode 216.

For example, the endoscope camera 220 may be move to a portion in contact with a plurality of electrodes and may photograph the contacted portion.

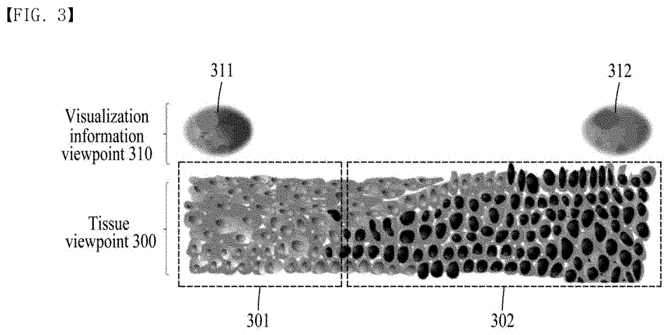

FIG. 3 is a drawing for explaining an electrical property image according to one embodiment of the present invention.

Specifically, FIG. 3 illustrates visualization information about electrical properties according to one embodiment of the present invention as compared with a tissue.

Referring to FIG. 3, according to one embodiment of the present invention, the screening apparatus may control a display to output a visualization information viewpoint 310 compared to a tissue viewpoint 300.

According to one embodiment of the present invention, the screening apparatus may control a display to output visualization information 311 and visualization information 312.

In the tissue viewpoint 300, normal tissue 301 and atypical tissue 302 may be represented.

In contrast, in the visualization information viewpoint 310, the visualization information 311 may represent the normal tissue 301, and the visualization information 312 may represent the atypical tissue 302.

For example, the atypical tissue 302 may be divided into mild, moderate, severe, in situ, and invasive according to the degree of deformity, and the distribution of the atypical tissue 302 may be a criterion for determining cervical cancer.

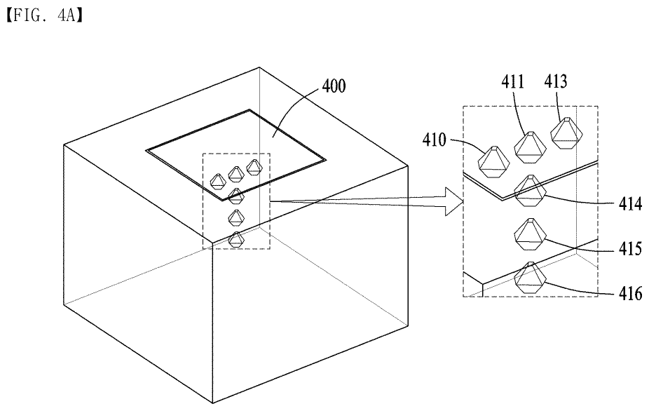

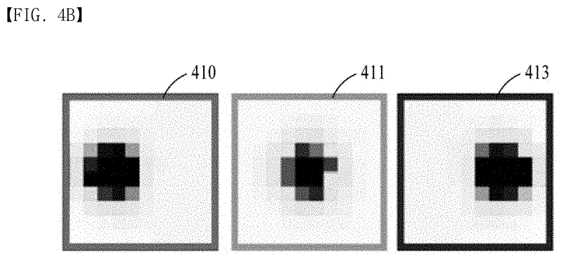

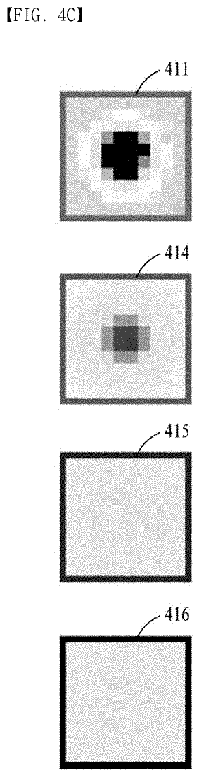

FIGS. 4A to 4C are drawings for explaining identification of the depth and location of atypical tissue based on electrical property images according to one embodiment of the present invention.

Referring to FIG. 4A, current or voltage may be applied to the inside of the human body through a portion 400 in contact with a plurality of electrodes, and current or voltage may be collected through the portion 400 in contact with the electrodes.

For example, a plurality of electrodes having an array structure described in FIG. 2 may be located at the portion 400.

According to one embodiment of the present invention, the screening apparatus may generate visualization information about a location 410, a location 411, a location 413, a location 414, a location 415, and a location 416. In this case, the location 410, the location 411, and the location 413 may be related to the location of atypical tissue, and the location 411, the location 414, the location 415, and the location 416 may be related to the depth of the atypical tissue.

Referring to FIG. 4B, the screening apparatus may generate visualization information corresponding to each location such as the location 410, the location 411, and the location 413.

The location 410 may be located relatively to the left, the location 411 may be located at the center, and the location 413 may be located relatively to the right.

Referring to FIG. 4C, the screening apparatus may generate visualization information corresponding to each depth such as the location 411, the location 414, the location 415, and the location 416.

The location 411 may represent a relatively large area, and the location 416 may represent a relatively small area.

That is, since the conductivity of an electrode decreases as a depth increases, the screening apparatus may display a narrow area. Since the conductivity of an electrode increases as a depth decreases, the screening apparatus may display a large area. Accordingly, in the screening apparatus, as the depth increases, displayed visualization information may not be relatively present.

Accordingly, according to the present invention, the degree of penetration by region and depth may be quantified by using a multi-channel array electrode.

FIGS. 5A and 5B are drawings for explaining identification of the location of atypical tissue based on electrical property images according to one embodiment of the present invention.

Specifically, FIG. 5A shows the locations of atypical tissue, and electrical properties measured at each location are shown in FIG. 5B.

Referring to FIG. 5A, case 500 in which atypical tissue is located at the middle of measurement electrodes may be illustrated. That is, conductivity generated from data measured by an electrode pair selected from measurement electrodes may best represent the electrical properties of the atypical tissue.

In addition, case 501 in which atypical tissue is located at a position corresponding to ½ of measurement array electrodes may be illustrated. That is, when electrodes corresponding to ½ of the measurement array electrodes are located above the atypical tissue and the remaining electrodes corresponding to ½ are located above normal tissue, conductivity generated from data measured by a selected electrode pair has the median value of the electrical properties of the atypical tissue and the electrical properties of the normal tissue, and the distribution of the atypical tissue and the normal tissue may be predicted from measurement data for each location of the measured electrode pair and sensitivity analysis.

In addition, a case in which atypical tissue is located at a position corresponding to ¼ of measurement array electrodes may be illustrated. That is, when electrodes corresponding to ¼ of the measurement array electrodes are located above the atypical tissue and the remaining electrodes corresponding to ¾ are located above normal tissue, conductivity generated from data measured by a selected electrode pair represents a value close to that of the normal tissue compared to case 501, and the distribution of the atypical tissue and the normal tissue may be predicted from measurement data for each location of the measured electrode pair and sensitivity analysis.

In addition, a case in which atypical tissue is located outside measurement array electrodes may be illustrated. That is, when the atypical tissue is located outside the measurement array electrodes, measured conductivity represents the electrical properties of normal tissue. Based on the image data of the endoscope, measurement must be repeated by moving to the adjacent area of the measurement area.

In addition, case 504 in which atypical tissue is located at the ½ edge of measurement array electrodes in a diagonal direction may be illustrated. That is, similar to case 501, when electrodes corresponding to ½ edge of the measurement array electrodes are located above the atypical tissue irrespective of direction and the remaining electrodes corresponding to ½ are located above normal tissue, similar to case 501, conductivity generated from data measured by the selected electrode pair has the median value of the electrical properties of the atypical tissue and the electrical properties of the normal tissue, and the distribution of the atypical tissue and the normal tissue may be predicted from measurement data for each location of the measured electrode pair and sensitivity analysis.

In addition, case 505 in which atypical tissue is located outside measurement array electrodes in a diagonal direction may be illustrated. That is, when the atypical tissue is located outside the measurement array electrodes, measured conductivity represents the electrical properties of normal tissue. Based on the image data of the endoscope, measurement must be repeated by moving to the adjacent area of the measurement area.

Referring to FIG. 5B, the horizontal axis of the graph indicates types according to the locations of atypical tissue based on the location of the measurement array electrodes shown in FIG. 5A, and the vertical axis indicates electrical properties (conductivity).

Type 510 may correspond to image 500, type 511 may correspond to image 501, type 512 may correspond to image 502, and type 513 may correspond to image 503, type 514 may correspond to image 504, and type 515 may correspond to image 505.

According to the graph, as the area where the location of atypical tissue overlaps measurement array electrodes increases, electrical properties due to the atypical tissue is highly reflected, and the distribution of the atypical tissue and the normal tissue in a corresponding region may be predicted through the measurement value and sensitivity analysis for each position of the array electrodes.

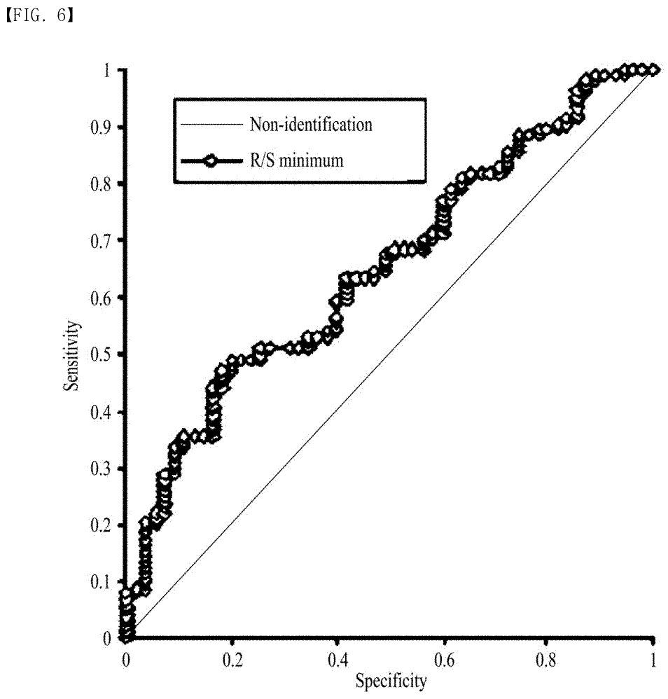

FIG. 6 is a graph for explaining measurement sensitivity versus specificity according to one embodiment of the present invention.

Specifically, FIG. 6 illustrates a receiver operating characteristic (ROC) curve obtained by analyzing sensitivity versus specificity of diagnosis through the present technology.

Referring to FIG. 6, in the graph, the horizontal axis represents specificity, and the vertical axis represents sensitivity. In this case, specificity and sensitivity are highly evaluated above the standard, meaning that the possibility of discrimination for atypical tissue is high.

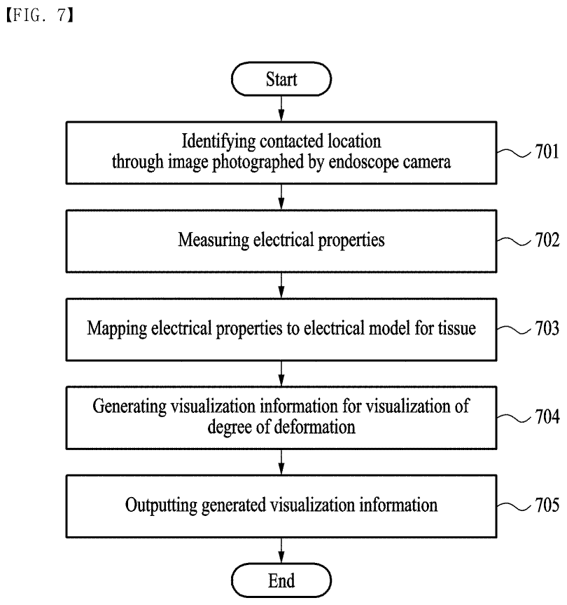

FIG. 7 is a flowchart for explaining a method of operating a screening apparatus according to one embodiment of the present invention.

Referring to FIG. 7, according to the method of operating a screening apparatus, in step 701, a contacted location may be identified through an image photographed by the endoscope camera.

That is, according to the method of operating a screening apparatus, a location in contact with a plurality of electrodes may be identified from an image photographed using an endoscope camera included in a probe inserted into the human body.

According to the method of operating a screening apparatus, in step 702, electrical properties may be measured.

That is, according to the method of operating a screening apparatus, the electrical properties of a specific portion may be measured by applying current or voltage through any one electrode of a specific electrode pair among a plurality of electrodes and collecting current or voltage transmitted through the specific portion through the remaining one electrode.

For example, the specific portion may include at least one of the vagina, the cervix, the rectum, the esophagus, and the stomach.

According to the method of operating a screening apparatus, in step 703, electrical properties may be mapped to an electrical model for a tissue.

That is, according to the method of operating a screening apparatus, measured electrical properties may be mapped to an electrical model corresponding to the electrical properties of normal tissue.

According to the method of operating a screening apparatus, in step 704, visualization information for visualizing the degree of deformation may be generated.

That is, according to the method of operating a screening apparatus, visualization information that represents the degree of deformation through difference in color, shade, shape, range, or distribution may be generated.

According to the method of operating a screening apparatus, in step 705, generated visualization information may be output.

That is, according to the method of operating a screening apparatus, a display may be controlled to output any one of the location, depth, or degree of progression of atypical tissue by controlling the display to output the degree of deformation of a tissue through difference in color, shade, shape, range, or distribution.

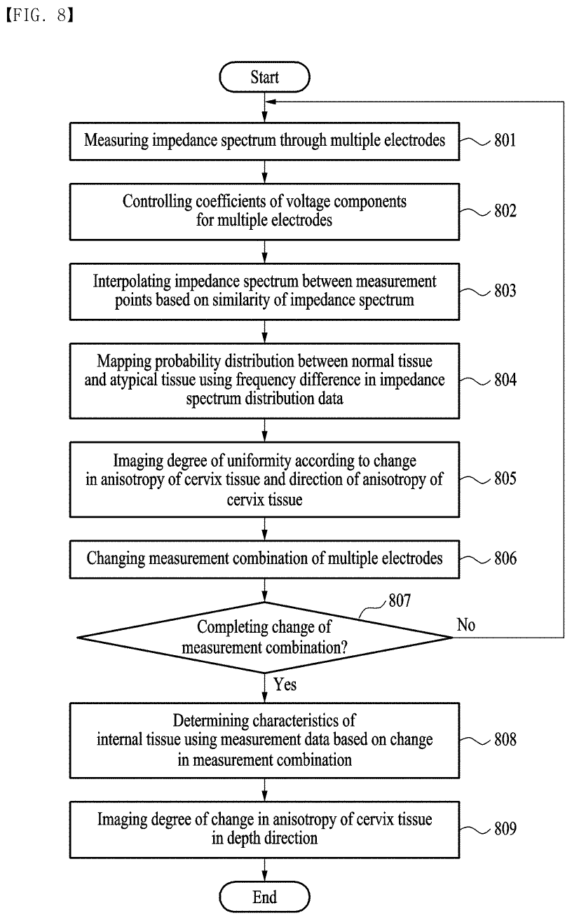

FIG. 8 is a flowchart for explaining a method of operating a screening apparatus according to one embodiment of the present invention.

Referring to FIG. 8, according to the method of operating a screening apparatus, a greater number of impedance spectrum measurement points than installed electrodes may be formed based on the geometric position of an injection current channel and a voltage measurement channel, and a map with improved resolution may be provided by generating interpolated data based on sensitivity measurement analysis between the measurement points by using the similarity between the positions of impedance spectra measured at each measurement point.

Referring to FIG. 8, in step 801, an impedance spectrum is measured through a plurality of electrodes.

Step 801 includes a step of supplying any one of current and voltage to any one electrode of a plurality of electrodes located at a probe and collecting any one of current and voltage through the remaining electrode to measure an impedance spectrum for a plurality of measurement points.

In step 802, the coefficients of voltage components for a plurality of electrodes are controlled.

Step 802 includes a step of determining a measurement sensitivity distribution for the physical properties of a region including the entire array electrode in consideration of the location and structure of a selected electrode pair.

In addition, step 802 includes a step of controlling the coefficients of voltage components for electrodes based on determined measurement sensitivity.

More specifically, step 802 may include a step of reducing the coefficients of voltage components for electrodes corresponding to the peripheral region of a measurement point among a plurality of electrodes and having relatively low measurement sensitivity, and a step of increasing the coefficients of voltage components for electrodes corresponding to the central region of a measurement point among a plurality of electrodes and having relatively high measurement sensitivity.

Accordingly, according to the present invention, in the lower region of the probe, information on a normal region and a dysplasia region may be integrally determined.

In step 803, an impedance spectrum between measurement points is interpolated based on the characteristics and similarity of the impedance spectrum of a tissue.

Step 803 may include a step of generating data for an impedance spectrum between a first measurement point and a second measurement point based on the similarity between the impedance spectrum of the first measurement point and the impedance spectrum of the second measurement point.

That is, according to the method of operating a screening apparatus, data on the impedance spectrum of a portion where the electrode is positioned or a portion where the electrode is not positioned may also be obtained.

Accordingly, according to the present invention, spatial resolution may be improved by interpolating data between measurement points based on the similarity between an impedance spectrum obtained at a measurement point and an impedance spectrum obtained at a surrounding measurement area.

In step 804, a probability distribution between normal tissue and atypical tissue may be mapped using frequency difference in impedance spectrum distribution data.

Step 804 may include a step of generating impedance spectrum distribution data using a measured impedance spectrum in step 801 and step 803.

In addition, in step 804, the distribution of normal tissue and atypical tissue may be probabilistically mapped using the frequency difference between impedance spectra in impedance spectrum distribution data.

In this case, the mapping data may be data on the distribution map of the normal tissue and the atypical tissue shown in FIG. 3.

In step 805, the degree of uniformity is imaged according to the anisotropy change and direction of a cervix tissue.

Step 805 includes a step of imaging the degree of uniformity according to the anisotropy impedance change and direction of a cervix tissue based on impedance spectrum distribution data.

In step 806, the measurement combination of a plurality of electrodes is changed.

Step 806 may include a step of supplying voltage or current through a first electrode among a plurality of electrodes, supplying voltage or current through a second electrode in measurement combination measuring the induced voltage or current with the second electrode, a third electrode, and a fourth electrode, and changing to measurement combination measuring with the first electrode, the third electrode, and the fourth electrode.

That is, step 806 may include a step of deriving a plurality of measurement combinations while changing the measurement interval between a plurality of electrodes, and changing a measurement combination according to the derived measurement combinations.

For example, when voltage or current is supplied through a first electrode and voltage or current is measured through a third electrode, a second electrode located between the first electrode and the third electrode may be skipped.

In addition, when the second electrode is skipped, since current or voltage is spatially spread more widely inside a measurement target, the degree to which the voltage decreases is relatively small.

Accordingly, when the difference between the maximum voltage and the minimum voltage increases relatively and a voltage range increases, since the number of voltages capable of being distinguished for noise increases, image quality may be improved.

However, as the number of skipped electrodes decreases, sensitivity for change in an impedance spectrum increases. Accordingly, when an image is restored, an integrated high-resolution image may be restored in consideration of a sensitivity distribution according to the locations of several electrode pairs.

In step 807, it is determined whether measurement combinations have been changed.

Step 807 includes a step of determining whether derivable measurement combinations have been changed based on a plurality of electrodes.

In this case, when all of measurement combinations have been used to measure an impedance spectrum, step 808 proceeds. When measurement combinations to be used to measure an impedance spectrum remain, the process returns to step 801 and steps 801 to 806 are performed again.

In step 808, the characteristics of an internal tissue are determined using measurement data based on change in measurement combination.

Step 808 includes a step of checking the characteristics of an internal tissue by unwrapping the layer of a measurement target from data measured according to an interval in electrode combination.

That is, according to the method of operating a screening apparatus, depth information on the inside of a measurement target may be obtained based on a plurality of impedance spectra.

For example, according to the method of operating a screening apparatus, an impedance spectrum between a first electrode and a second electrode may be measured, an impedance spectrum between the first electrode and a third electrode may be measured when the distance between the third electrode and the first electrode is longer than the distance between the second electrode and the first electrode, and internal depth information for a region located between the first electrode and the third electrode may be obtained from the difference between the measured impedance spectra.

In step 809, the degree of change in the anisotropy of a cervix tissue in the depth direction is imaged.

Step 809 may include a step of imaging the degree of change in the anisotropy of a cervix tissue in the depth direction by imaging electrical properties reflecting internal depth information obtained in step 808.

Accordingly, the present invention may increase the probability of detecting cervical dysplasia and cervical cancer by improving the efficiency of designating a biopsy area by providing all electrical property values and a map of electrical property change by area.

The methods according to the embodiments of the present invention may be implemented in the form of a program command that can be executed through various computer means and recorded in a computer-readable medium. The computer-readable medium can store program commands, data files, data structures or combinations thereof.

The program commands recorded in the medium may be specially designed and configured for the present invention or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands.

Examples of the program commands include machine language code created by a compiler and high-level language code executable by a computer using an interpreter and the like. The hardware devices described above may be configured to operate as one or more software modules to perform the operations of the embodiments, and vice versa.

Although the present invention has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

1. A screening apparatus using a multi-channel array electrode probe, comprising:
a probe inserted inside of a human body and comprising a plurality of electrodes in contact with any one portion of the inside and an endoscope camera for photographing the portion;
a location identifier for identifying a location in contact with the electrodes in an image photographed by the endoscope camera;
an electrical property measuring device for measuring electrical properties of the identified location;
a mapping processor for mapping the measured electrical properties to an electrical model for the internal tissue; and
a visualization information generator for, based on the mapping results, generating visualization information for visualization of a degree of deformation of the tissue with respect to the measured electrical properties.

2. The screening apparatus according to claim 1, wherein, based on similarity between an impedance spectrum of a first measurement point and an impedance spectrum of a second measurement point among a plurality of measurement points located under the probe, the electrical property measuring device generates data about an impedance spectrum between the first measurement point and the second measurement point.

3. The screening apparatus according to claim 2, wherein the mapping processor generates impedance spectrum distribution data based on a plurality of impedance spectra related to the measured electrical properties, and maps a probability distribution between normal tissue and atypical tissue to an electrical model for the internal tissue using frequency difference in the impedance spectrum distribution data.

4. The screening apparatus according to claim 1, wherein the electrical property measuring device determines a measurement sensitivity distribution for the portion according to a location of an electrode pair selected from the electrodes.

5. The screening apparatus according to claim 4, further comprising a probe controller for determining an electrode pair for collecting any one of current and voltage from the electrodes based on the determined measurement sensitivity distribution.

6. The screening apparatus according to claim 5, wherein, to adjust a distance between the electrodes, the probe controller changes a combination of electrodes constituting the electrode pair, and integrates and analyzes a plurality of measurement data to increase measurement sensitivity.

7. The screening apparatus according to claim 6, wherein the probe controller controls the probe to measure an electrical impedance spectrum by selecting any one electrode from the electrode pair and supplying any one of current and voltage to the electrode, and by collecting any one of current and voltage through the remaining electrode of the electrode pair.

8. The screening apparatus according to claim 1, wherein the electrical property measuring device non-invasively measures electrical properties of the internal tissue by measuring an electrical impedance spectrum of the internal tissue through the electrodes.

9. The screening apparatus according to claim 1, wherein the portion comprises a portion accessible through an orifice of the human body, and
the accessible portion comprises at least one of a vagina, a cervix, a rectum, an esophagus, and a stomach.

10. The screening apparatus according to claim 1, further comprising a display controller for controlling a display to output the generated visualization information,
wherein the generated visualization information represents a degree of deformation of the tissue through difference in color, shade, shape, range, or distribution.

11. The screening apparatus according to claim 10, wherein the display controller controls the display to output any one of a location, a depth, or a degree of progression of atypical tissue (dysplasia) through the difference in color, shade, shape, range, or distribution.

12. A method of operating a screening apparatus using a multi-channel array electrode probe, wherein the screening apparatus comprises a probe inserted inside of a human body and comprising a plurality of electrodes in contact with any one portion of the inside and an endoscope camera for photographing the portion, and
the method comprises a step of identifying, in a location identifier, a location in contact with the electrodes in an image photographed by the endoscope camera;
a step of measuring, in an electrical property measuring device, electrical properties of the identified location;
a step of mapping, in a mapping processor, the measured electrical properties to an electrical model for the internal tissue; and
a step of generating, in a visualization information generator, visualization information for visualization of a degree of deformation of the tissue with respect to the measured electrical properties based on the mapping results.

13. The method according to claim 12, wherein the step of measuring electrical properties comprises a step of determining a measurement sensitivity distribution for the portion according to a location of an electrode pair selected from the electrodes;
a step of determining, in a probe controller, an electrode pair for collecting any one of current and voltage from the electrodes based on the determined measurement sensitivity distribution; and
a step of changing, in the probe controller, a combination of electrodes constituting the electrode pair to adjust a distance between the electrodes, and integrating and analyzing a plurality of measurement data to increase measurement sensitivity.

14. The method according to claim 13, wherein the step of determining an electrode pair for collecting any one of current and voltage comprises a step of selecting any one electrode from the electrode pair and supplying any one of current and voltage to the electrode; and
a step of controlling the probe to measure an electrical impedance spectrum by collecting any one of current and voltage through the remaining electrode of the electrode pair.

15. The method according to claim 12, further comprising a step of controlling, in a display controller, a display to output the generated visualization information,
wherein the generated visualization information represents a degree of deformation of the tissue through difference in color, shade, shape, range, or distribution.

16. The method according to claim 15, wherein the step of controlling a display to output the generated visualization information comprises a step of controlling the display to output any one of a location, a depth and a degree of progression of atypical tissue (dysplasia) through the difference in color, shade, shape, range, or distribution.
